(19) United States
(12) Patent Application Publication
(10) Pub. No.: US 2021/0386569 A1
(43) Pub. Date: Dec. 16, 2021

(54) CONSTRAINING MECHANISMS AND ASSOCIATED METHODS

(71) Applicant: W. L. Gore & Associates, Inc., Newark, DE (US)

(72) Inventor: Jerry J. Stastka, Flagstaff, AZ (US)

(21) Appl. No.: 17/282,907

(22) PCT Filed: Oct. 4, 2019
(86) PCT No.: PCT/US2019/054630

§ 371 Date: Apr. 5, 2021

Related U.S. Application Data
(60) Provisional application No. 62/741,944, filed on Oct. 5, 2018.

(51) Int. Cl. A61F 2/962; A61F 2/82

(52) U.S. Cl. CPC A61F 2/962; A61F 2002/9511; A61F 2/82

(57) ABSTRACT

Various aspects of the present disclosure are directed toward apparatuses, systems, and methods that include an implantable medical device and a constraining mechanism configured to releasably constrain the implantable medical device in a delivery configuration. The apparatuses, systems, and methods may also include a lock line arranged through the constraining mechanism to tenable release of the constraining mechanism.

### CROSS-REFERENCE TO RELATED APPLICATION

This application is a national phase application of PCT Application No. PCT/US2019/054630, internationally filed on Oct. 4, 2019, which claims the benefit of Provisional Application No. 62/741,944, filed Oct. 5, 2018, which are incorporated herein by reference in their entireties for all purposes.

### FIELD

The present disclosure relates to apparatuses, systems, and methods that include constructing coverings used in delivery of implantable medical devices. More specifically, the present disclosure relates to apparatuses, systems, and methods that include constructing coverings for constraining an expandable device during device delivery.

### BACKGROUND

Stents and stent-grafts may be utilized to radially support a variety of tubular passages in the body, including arteries, veins, airways, gastrointestinal tracts, and biliary tracts. The preferred method of placing these devices has been to use specialized delivery systems to precisely place and deploy a device at the site to be treated. These delivery systems allow the practitioner to minimize the trauma and technical difficulties associated with device placements. Attributes of delivery systems include: low profile; ability to pass through introducer sheaths; ability to negotiate tortuous vasculature, smoothly and atraumatically; protection of constrained devices; and ability to accurately position and deploy the device.

Stents or stent-grafts may be deployed and plastically deformed by using an inflatable balloon (e.g., balloon expandable stents) or to self-expand and elastically recover (e.g., self expandable devices) from a collapsed or constrained delivery diameter to an expanded and deployed diameter. Some stents are designed to elastically recover by being manufactured at their functional diameter out of a material that has elastic recovery properties, and then radially compressed to be mounted on a delivery catheter.

These stent and stent-graft devices may be held, compressed, or constrained in the delivery configuration prior to and during delivery to a target location.

### SUMMARY

In one example (“Example 1”), a delivery system includes an implantable medical device; a constraining mechanism including interlocking loops configured to releasably constrain the implantable medical device in a delivery configuration, and a lock line arranged through a portion of the interlocking loops and configured to be withdrawn to enable release of the constraining mechanism.

In another example (“Example 2”), further to the delivery system of Example 1, each loop of the interlocking loops includes at least two strands forming the loop, and wherein one strand of the loop overlaps with a strand of an adjacent loop to form an interlocking loop.

In another example (“Example 3”), further to the delivery system of any one of Examples 1-2, the interlocking loops include at least two strands arranged in a warp knit having multiple knot rows spaced around a circumference of the implantable medical device.

In another example (“Example 4”), further to the delivery system of Example 3, the lock line is arranged through one row of the interlocking loops.

In another example (“Example 5”), further to the delivery system of Example 3, the lock line is arranged through both rows of the loops.

In another example (“Example 6”), further to the delivery system of any one of Examples 2-5, when one of the knot rows is disrupted, the constraining mechanism unravels and is remotely removable when a force is applied to a deployment line.

In another example (“Example 7”), further to the delivery system of any one of Examples 2-6, the lock line is arranged in at least one of the knot rows to prevent the knot row from unraveling.

In another example (“Example 8”), further to the delivery system of Example 7, the lock line comprises a linchpin.

In another example (“Example 9”), further to the delivery system of Example 8, the linchpin is removable to allow a user to selectively unravel the at least one knot row.

In another example (“Example 10”), further to the delivery system of Example 7, the lock line comprises an adhesive on an exterior surface of the lock line to increase friction between the interlocking loops.

In another example (“Example 11”), further to the delivery system of any one of Examples 4-5, the lock line is configured to resist deployment when a ratio of a deployed diameter of the device to a delivery diameter of the device is less than 0.3.

In another example (“Example 12”), further to the delivery system of any one of Examples 7-10, the lock line is arranged through each of the knot rows to allow for controlled release of each of the knot rows.

In another example (“Example 13”), further to the delivery system of any one of Examples 1-12, the lock line is a first lock line, and wherein the system further comprises a second lock line arranged through another portion of interlocking loops.

In another example (“Example 14”), further to the delivery system of Example 13, the second lock line is spaced a distance from the first lock line around the circumference of the constraining mechanism.

In another example (“Example 15”), further to the delivery system of Example 13, the first lock line is configured to release a first portion of the interlocking loops and the second lock line is configured to release a second portion of the interlocking loops.

In one example (“Example 16”), a delivery system includes an implantable medical device; a constraining mechanism configured to constrain the implantable medical device to a delivery configuration, and a lock line configured to increase friction between interlocking loops of at least one knot row to maintain the constraining mechanism in the delivery configuration.

In another example (“Example 17”), further to the delivery system of Example 16, the lock line is removed from the delivery system by applying a force to the lock line, and wherein removal of the lock line releases the constraining mechanism.

In another example (“Example 18”), further to the delivery system of any one of Examples 16-17, removal of the lock line releases each loop of the interlocking loops sequentially.

In one example (“Example 19”), a method for using a delivery system includes arranging a medical device in a delivery configuration using a constraining mechanism, the constraining mechanism including interlocking strands forming a knot row and a lock line extending through at least a portion of the knot row; positioning the delivery system at a desired treatment location in the body of a patient while the implantable medical device is in the delivery configuration; and applying a force to the lock line to release the constraining mechanism and deploy the medical device.

In another example (“Example 20”), further to the method of Example 19, applying the force to the lock line decreases friction between the interlocking strands of the knot row to sequentially unravel the knot row.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a top plan view of a delivery system including a catheter with a constraining mechanism, in accordance with an embodiment;

FIG. 2 is a side view of an implantable medical device including a constraining mechanism, in accordance with an embodiment;

FIG. 3 is a schematic view of interlocking strands of the constraining mechanism, in accordance with an embodiment;

FIG. 4 is a schematic view of interlocking strands of the constraining mechanism, in accordance with an embodiment;

FIG. 5 is a schematic view of interlocking strands of the constraining mechanism having multiple lock lines, in accordance with an embodiment;

FIGS. 6A-6C are end views of the constraining mechanism showing example knot row positions, in accordance with an embodiment.

FIGS. 7A-7B are images of a delivery system in a delivery configuration and a semi-deployed configuration, respectively, in accordance with an embodiment;

As the terms are used herein with respect to ranges of measurements “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error, differences in measurement and/or manufacturing equipment calibration, human error in reading and/or setting measurements, adjustments made to optimize performance and/or structural parameters in view of differences in measurements associated with other components, particular implementation scenarios, imprecise adjustment and/or manipulation of objects by a person or machine, and/or the like.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### DETAILED DESCRIPTION

Persons skilled in the art will readily appreciate that various aspects of the present disclosure can be realized by any number of methods and apparatus configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Various aspects of the present disclosure are directed toward apparatuses, systems, and methods that include forming or manufacturing a constraining mechanism. The constraining mechanisms are configured to hold, compress, or constrain an implantable medical device (e.g., a stent, stent-graft, balloon, or other expandable medical device) in a delivery configuration prior to and during delivery to a target location. In certain instances, the constraining mechanism includes one or more fibers.

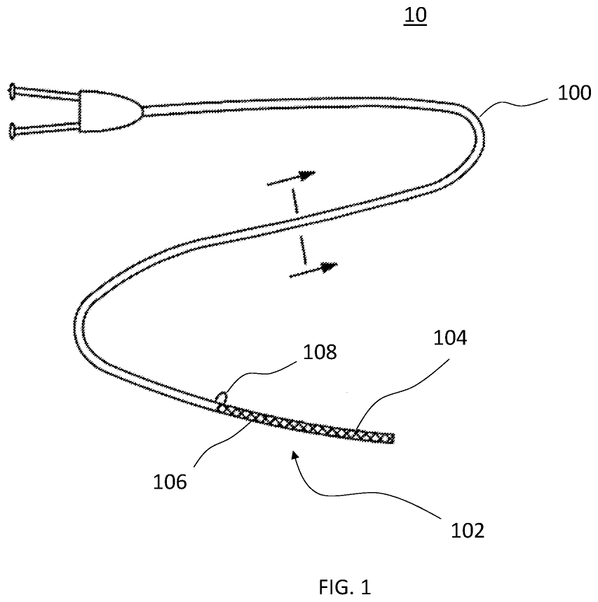

FIG. 1 is a top plan view of a delivery system 10 including a catheter 100 with a removable constraint 102, according to some embodiments. As shown in FIG. 1, the removable constraint 102 is configured to constrain an implantable medical device 104 to a delivery configuration. The removable constraint 102 may include one or more fibers 106 arranged about the device 104 to maintain the removable constraint 102 in a constrained configuration.

The removable constraint 102 is arranged along a length of the device 104. The removable constraint 102 is also circumferentially arranged about the device 104 and may substantially cover the device 104 for delivery. The one or more fibers 106 may be arranged within a lumen (not shown) of the catheter 100 and extend toward a proximal end of the catheter 100 that is arranged external to a patient during delivery of the device 104. The one or more fibers 106 include a proximal end 108 that a user may apply tension to in order to release the removable constraint 102 and deploy the device 104.

In certain instances, the one or more fibers 106 release similar to a rip cord such that interlocking portions (e.g., overlapping fibers or knots) sequentially release along the length of the device 104. As is explained in greater detail below, the removable constraint 102 is formed by interlocking together the one or more fibers 106 directly on the device 104. The device 104 may be a stent, stent-graft, a balloon, or a similar device.

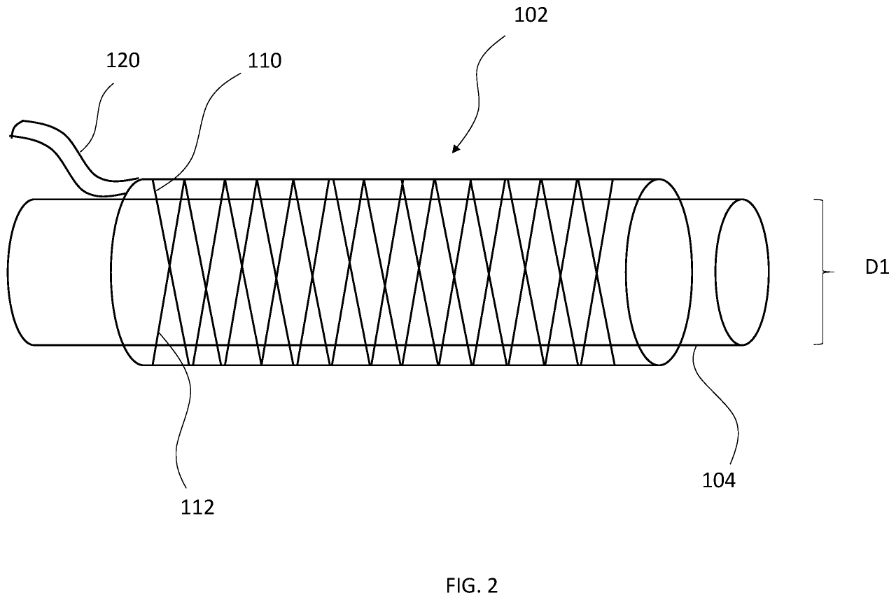

FIG. 2 is a side view of the device 104 including the constraining mechanism 102, in accordance with an embodiment. As shown, the device 104 includes a delivery diameter D1 and a deployed diameter D2 (not shown) that is larger than the delivery diameter D1. The removable constraint 102 is attached to the device 104 at its delivery diameter D1. As shown, the constraining mechanism 102 includes at least two interlocking strands in the form of a warp knit. For example, the constraining mechanism 102 may include a first interlocking strand 110 and a second interlocking strand 112. The constraining mechanism 102 may include a lock line 124 configured to release the constraining mechanism 102 and release the device 104 from the delivery diameter D1 to the deployed diameter D2 in response to a force applied to the lock line 124. In other terms, when the lock line 124 is removed from the constraining mechanism 102, the constraining mechanism 102 is released. decreases friction between the interlocking loops to sequentially unravel the at least one knot row.

The device 104 may have a desired deployed diameter D2 from about 5 mm-15 mm, or 6 mm-9 mm, or 6 mm-12 mm, for example, and a delivery diameter D1 that is less than the deployed diameter D2. For example, in some instances, a ratio of the delivery diameter D1 of the device 104 to the deployed diameter D2 (not shown) of the device 104 is less than about 0.3, less than about 0.29, less than about 0.28, less than about 0.27, or less than about 0.26. For reference, the term “diameter” is not meant to require a circular cross-section, and is instead to be understood broadly to reference a maximum transverse cross-sectional dimension of a device 104.

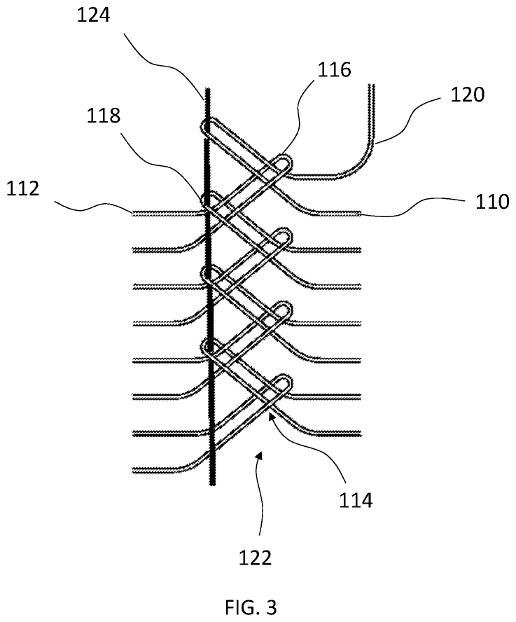

FIG. 3 is a schematic view of interlocking strands of the constraining mechanism 102, in accordance with an embodiment. The interlocking strands (e.g., the first and second interlocking strands 110, 112 as shown) are interwoven with one another to form at least one knot 114. As shown, the knot 114 is formed of interlocking loops formed from the first and second interlocking strands 110, 112. For example, the first interlocking loop 116 is formed by the first interlocking strand 110 and is interwoven with the second interlocking loop 118 formed by the second interlocking strand 112. This interlocking, looped configuration may be repeated to extend the longitudinal length of the device 104 (as shown in FIGS. 1, 2, and 7A-B) to form a knot row 122.

In some instances, the lock line 124 is arranged through the knot row 122 of the constraining mechanism 102. The lock line 124, in connection with the interlocking strands 110, 112, is configured to lessen ramping (or deployment angle) of the device 104 prior to being released. For example, the lock line 124 may be configured to lessen ramping of the device 104 prior to the knots 114 being released in sequence. The device 104 begins to expand to a larger diameter after release of the constraining mechanism 102.

As discussed in further detail below, the lock line 124 lessens ramping of the device 104 (which may lead to uncontrolled or undesired deployment) by maintaining a location of each of the knots 114, relative to the device 104, as the knots 114 are released in sequence. The lock line 124, in this manner, lessens undesired or pre-deployment of the device. In some instances, the lock line 124 can be a fiber, wire, rod, or other similar device that is capable of extending along the knot row 122.

As force is applied to the lock line 124 and the lock line 124 is removed from the constraining mechanism 102, each of the knots 114 may be released in sequence. The knots 114 may be released as the lock line 124 is withdrawn or the knots 114 may be released by applying tension to a deployment line 120, which is an end of one or both of the interlocking strands 110, 112. Removal of the lock line 124 decreases friction between the interlocking strands of the constraining mechanism 102 to sequentially unravel the knot row 122. Thus, when force is applied to the lock line 124, the constraining mechanism 102 is remotely removable by a user.

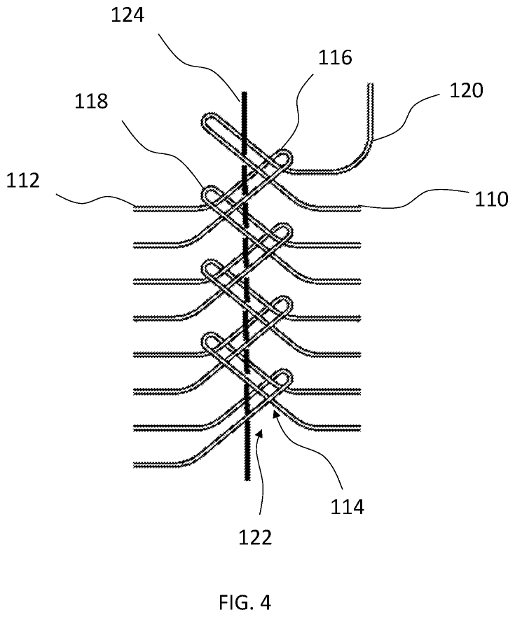

In some instances, the lock line 124 is arranged through one of the interlocking loops (e.g., either the first or second interlocking loop 116, 118), as shown in FIG. 3. In other instances, the lock line 124 can be arranged through both of the interlocking loops, as shown in FIG. 4. In certain instances, the lock line 124 is incorporated into at least one of the knots 114 to prevent the knot 114 from unraveling. Examples of suitable lock lines 124 can include linchpins, wires, metallic lines, fibers, and various adhesives. In certain instances, the lock line 124 is removable to allow a user to selectively unravel the respective knot 114. In some instances, the lock line 124 may be incorporated into each of the knots 114 for controlled release of all knots 114 of a respective knot row 122 (FIG. 3).

FIG. 5 is a schematic view of interlocking strands of the constraining mechanism 102 having multiple lock lines 124, in accordance with an embodiment. As shown, the knot row 122 can include more than one lock line 124. For example, the knot row 122 may include a first lock line 124a arranged through a first portion of the interlocking loops and a second lock line 124b arranged through a second portion of the interlocking loops. In certain instances, the first lock line 124a and the second lock line 124b longitudinally overlap. The first lock line 124a and the second lock line 124b may originate from a user end of the delivery system 10 that uses the constraining mechanism 102. In other instances, one of the first lock line 124a and the second lock line 124b may be coupled to or bifurcate from the other of the first lock line 124a and the second lock line 124b. The first lock line 124a may configured to release the first portion of the interlocking loops while the second lock line 124b is configured to release the second portion of interlocking loops. In some instances, the constraining mechanism 102 may include more than one knot row 122.

The knots 114 may be released as the lock lines 124a, 124b are withdrawn or the knots 114 may be released by applying tension to a deployment line 120, which is an end of one or both of the interlocking strands 110, 112. Removal of the lock lines 124a, 124b decreases friction between the interlocking strands of the constraining mechanism 102 to sequentially unravel the knot row 122. Thus, when force is applied to the lock line 124, the constraining mechanism 102 is remotely removable by a user.

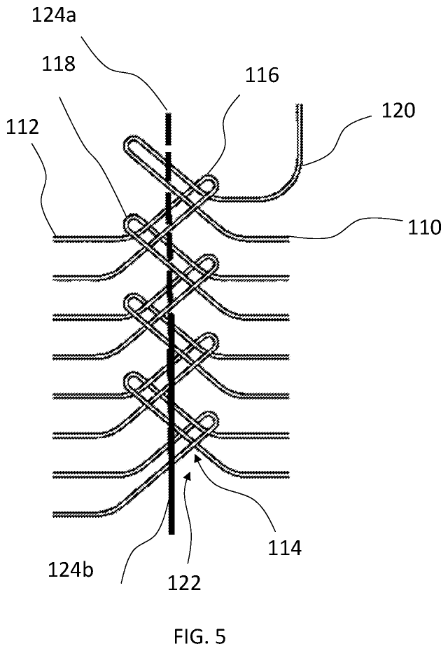

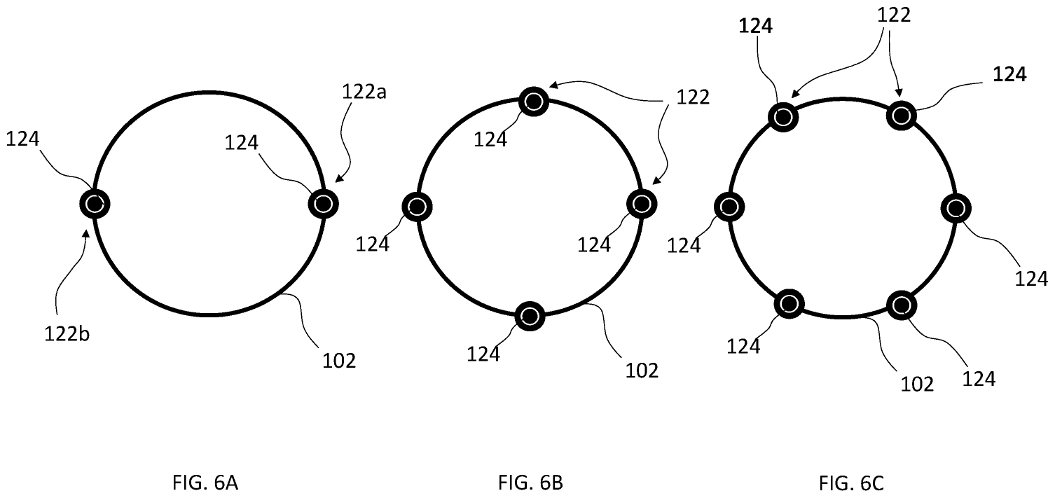

FIG. 6A is an end view of the device 104 including the constraining mechanism 102, according to an embodiment. As shown, the constraining mechanism 102 includes two knot rows. For example, the constraining mechanism 102 includes a first knot row 122a and a second knot row 122b spaced a distance from the first knot row 122a about the circumference of the constraining mechanism 102. In some instances, each of the knot row 122a, 122b includes a lock line 124 (e.g., a first lock line 124a and a second lock line 124b). For example, the first knot row 122a can include the first lock line 124a and the second knot row 122b can include the second lock line 124b.

FIGS. 6B and 6C are end views of the device 104 including the constraining mechanism 102, according to an embodiment. As shown, the constraining mechanism 102 can include more than two knot rows 122. For example, the constraining mechanism 102 can include four, six, or more knot rows 122 spaced about the circumference of the constraining mechanism 102 as desired. The loops in the knot rows 112 may be of different diameters in certain instances.

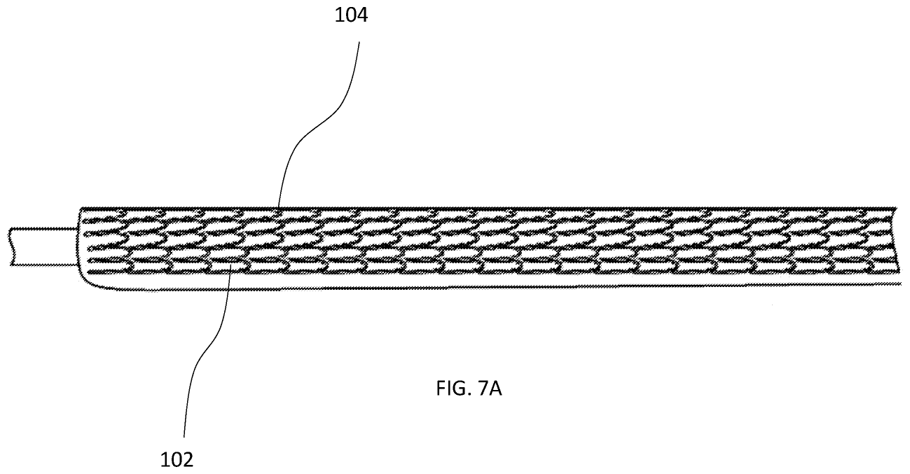

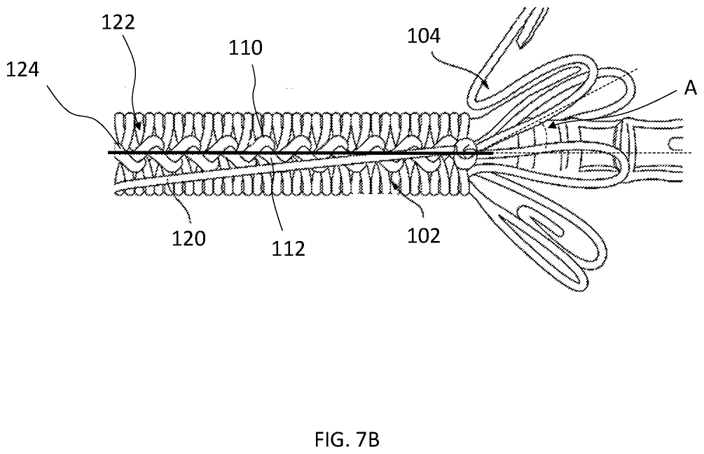

FIGS. 7A-7B are images of a delivery system in a delivery configuration and a semi-deployed configuration, respectively, in accordance with an embodiment. As shown in FIG. 7A, the removable constraint 102 is attached to the device 104 at its delivery diameter D1. During deployment, a force is applied to the lock line 124 to release the removable constraint 102 by unraveling the knot row 112, as shown in FIG. 7B. In certain instances, the knot row 112 may sequentially release, after the lock line 124 is removed, by applying a force to the deployment line 120. The device 104 is released to the deployed diameter D2 as the removable constraint 102 is released.

The lock line 124 can lessen ramping of the device 104 prior to being released. For example, the lock line 124 may lessen ramping of the device 104 prior to the knots of the knot row 122 being released in sequence. The device 104 begins to expand to a larger diameter after release of the constraining mechanism 102. The device 104 may be have an angle A between the portions held by the constraining mechanism 102 and portions that have been expanded or are beginning to expand. Due to the angle A and the device 104 expending a force to deploy to the deployed diameter D2, prior devices may shift due to ramping of the device 104. The lock line 124, however, resists spontaneous deployment that can be magnified by ramping of the device 104 with or without application of a radial force by the compressed stent.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A delivery system comprising:
an implantable medical device;
a constraining mechanism including interlocking loops configured to releasably constrain the implantable medical device in a delivery configuration, and
a lock line arranged through a portion of the interlocking loops and configured to be withdrawn to enable release of the constraining mechanism.

2. The delivery system of claim 1, wherein each loop of the interlocking loops includes at least two strands forming the loop, and wherein one strand of the loop overlaps with a strand of an adjacent loop to form an interlocking loop.

3. The delivery system of claim 1, wherein the interlocking loops include at least two strands arranged in a warp knit having multiple knot rows spaced around a circumference of the implantable medical device.

4. The delivery system of claim 3, wherein the lock line is arranged through one row of the interlocking loops.

5. The delivery system of claim 3, wherein the lock line is arranged through both rows of the loops.

6. The delivery system of claim 1, wherein when one of the knot rows is disrupted, the constraining mechanism unravels and is remotely removable when a force is applied to a deployment line.

7. The delivery system of claim 2, wherein the lock line is arranged in at least one of the knot rows to prevent the knot row from unraveling.

8. The delivery system of claim 7, wherein the lock line comprises a linchpin.

9. The delivery system of claim 8, wherein the linchpin is removable to allow a user to selectively unravel the at least one knot row.

10. The delivery system of claim 7, wherein the lock line comprises an adhesive on an exterior surface of the lock line to increase friction between the interlocking loops.

11. The delivery system of claim 4, wherein the lock line is configured to resist deployment when a ratio of a deployed diameter of the device to a delivery diameter of the device is less than 0.3.

12. The delivery system of claim 7, wherein the lock line is arranged through each of the knot rows to allow for controlled release of each of the knot rows.

13. The delivery system of claim 1, wherein the lock line is a first lock line, and wherein the system further comprises a second lock line arranged through another portion of interlocking loops.

14. The delivery system of claim 13, wherein the second lock line is spaced a distance from the first lock line around the circumference of the constraining mechanism.

15. The delivery system of claim 13, wherein the first lock line is configured to release a first portion of the interlocking loops and the second lock line is configured to release a second portion of the interlocking loops.

16. A delivery system comprising:
an implantable medical device;
a constraining mechanism configured to constrain the implantable medical device to a delivery configuration, and
a lock line configured to increase friction between interlocking loops of at least one knot row to maintain the constraining mechanism in the delivery configuration.

17. The delivery system of claim 16, wherein the lock line is removed from the delivery system by applying a force to the lock line, and wherein removal of the lock line releases the constraining mechanism.

18. The delivery system of claim 16, wherein removal of the lock line releases each loop of the interlocking loops sequentially.

19. A method for using a delivery system, the method comprising:
arranging a medical device in a delivery configuration using a constraining mechanism, the constraining mechanism including interlocking strands forming a knot row and a lock line extending through at least a portion of the knot row;
positioning the delivery system at a desired treatment location in the body of a patient while the implantable medical device is in the delivery configuration; and
applying a force to the lock line to release the constraining mechanism and deploy the medical device.

20. The method of claim 19, wherein applying the force to the lock line decreases friction between the interlocking strands of the knot row to sequentially unravel the knot row.
